(19) United States
(12) Patent Application Publication
(10) Pub. No.: US 2026/0106043 A1
(43) Pub. Date: Apr. 16, 2026

(54) Air longevity

(71) Applicant: Anuthep Benja-Athon, Manhasset, NY (US)

(72) Inventor: Anuthep Benja-Athon, Manhasset, NY (US)

(73) Assignee: Anuthep Benja-Athon, Manhasset, NY (US)

(21) Appl. No.: 18/831,285

(22) Filed: Oct. 15, 2024

(51) Int. Cl. G16H 50/70; G06F 3/0481; G06T 11/20

(52) U.S. Cl. CPC G16H 50/70; G06F 3/0481; G06T 11/26

(57) ABSTRACT

A plurality of interactive comparative graphs for expressing and demonstrating the plurality of interactive electronic templates-expressed null relations and causal relations producers'data relating to same common diagnoses, diseases and disorders and relational health-healthcare data, and beneficial effects.

### FIELD OF THE INVENTION

The networked devices proving the causal relations of chemicals and diseases.

### BACKGROUND OF THE INVENTION

Evolving the evolution, an abiotic intelligence-rendered billions of networked smartphones, laptops, desktops and servers creating the largest digital laboratory wherein billions of people's and animals in situ and in vivo data directly and indirectly affecting lives and longevity of billions of people and animals on earth.

In the food and pharmaceutical industries, so much have been achieved and delivering excellent products for billions of people and house and farm animals. However, among producers'data of foods and related items and pharmaceuticals for people and animals, a small percentage of foods and related items and pharmaceuticals have been tested, examined and analyzed in laboratories within relatively short times and small samples of subjects.

Consequently, all people heretofore neither know nor understand the short and long terms effects of most foods and related items and pharmaceuticals which are being ingested and exposed with good, nutritious, detrimental, adverse and useless aka null effects, such as, but not limited to, inflammations and acute, subacute and chronic diseases, disorders and sequelae, such as, but not limited to nervous system and cardiovascular diseases and disorders, or benefits in years, decades and generations. Consequently, the aforementioned null relations and causal relations, and intangible losses and losses in the $billions per annum, are heretore unknown to people all over the world.

### SUMMARY OF THE INVENTION

The abiotic intelligence-created networked communication devices comprising a plurality of interactive electronic templates for creating, compiling thence expressing and demonstrating the null relations and causal relations of the producers'data relating to the cumulative incidences, prevalences and durations of the same common diagnoses, diseases and disorders and sequelae, relational health-healthcare data, and beneficial effects in the plurality of interactive comparative graphs for enlightening all people on the null relations and causal relations.

### DESCRIPTION OF THE PREFERRED EMBODIMENTS

The Periodic Table comprised 118 elements forming compounds and matters including all people and animals on earth. In other words, in all people and animals, their genes, DNA, RNA, amino acids and all compounds, matters, functions, products and outcomes in the AIR Longevity, are created by evolution from the elements in the Periodic Table. Thus, the biochemistry and chemistry in the former and latter are analogous to trillions pairs of biological and biochemical locks and keys in the evolution of the aforementioned in the Universe. Therefore, influent by external environments and factors, there are specific and precise correlations and causal relations the elements, issues and matters which are addressed in the present invention.

As an analogy, biological and biochemical malfunctions such as, but not limited to, defective genes and the defective products relating to ingestions and exposures to carcinogenes, initiators and promoters are a pathologic basis of thousands of inflammations, acute, subacute and chronic diseases, disorders and sequelae such as, but not limited to, the central, peripheral and autonomic nervous systems such as, but not limited to, senile dementia, pain and Alzheimer's disease, and cardiovascular diseases such as, but not limited to, heart diseases which are still the dominant cause of death in the USA.

In a nutshells, the aforementioned paradigm and, by default, the AIR Longevity applied to billions people's abiotic intelligence-created networked communication devices for creating, analyzing, researching, establishing and expressing the cumulative incidences, prevalences of common expressing the null relations and causal relations of the producers'data relating to the cumulative incidences, prevalences and durations of the same common denominators comprising diagnoses, diseases and disorders and sequelae, relational health-healthcare data, and beneficial effects inherent in the all people's, physicians'and scientists'and the animal's in situ and in vivo data, in and shared among the storage memories, and the plurality of interactive comparative graphs for expressing and demonstrating the null relations and causal relations for enlightening all people on the null relations and causal relations in and among the touchscreens and viewing screens of billions people's abiotic intelligence-created networked communication devices on earth.

In contrast to the aforementioned laboratories, the abiotic intelligence-networked communication devices comprising the billions abiotic intelligence-networked servers, smartphones, laptops and desktops, being the omnipresent and omnipotent digital laboratories 24/7 creating, establishing and expressing the null relations and causal relations in situ and in vivo all over the earth and functioning for minutes, hours, days, decades and centuries, comprising all people and animals and billions touchscreens and viewing screens, cameras, microphones, the processor units attaching to the storage memories storing the innovative computer programs including the commercial software and millions of the producers'data relating to the cumulative incidences, prevalences and durations of the same common denominators comprising same common diagnoses, same common diseases and same common disorders and same common sequelae, same common relational health-healthcare data, and beneficial effects inherent in all people's, physicians'and scientists'and animal's in situ and in vivo data on earth.

And said billions abiotic intelligence-networked servers, smartphones, laptops and desktops creating:
the plurality of interactive electronic templates creating the plurality of interactive electronic templates-expressed null relations and causal relations comprising the processes of the innovative computer programs instructing the processor units to creating:
a plurality of interactive electronic templates letting the producers inputting and compiling their producers'same data as compilations of producers'same data in and shared among the storage memories;
a plurality of interactive electronic templates getting and creating the compilations of producers'same data as interactive and selectable compilations of producers'same data in a plurality of interactive electronic templates;
a plurality of interactive electronic templates letting the people, physicians and scientists inputting and compiling the people's, physicians'and scientists'and the animal's in situ and in vivo data as compilations of in situ and in vivo data in and shared among the storage memories;
a plurality of interactive electronic templates getting and creating the compilations of in situ and in vivo data as interactive and selectable compilations of in situ and in vivo data in a plurality of interactive electronic templates in and shared among the storage memories; and
said plurality of interactive electronic templates letting the people, physicians and scientists selecting and relating the interactive and selectable compilations of the producers'data to interactive and selectable compilations of the same common denominators in the people's, physicians'and scientists'and the animal's in situ and in vivo data, in and shared among the storage memories wherein
said plurality of interactive electronic templates-expressed null relations and causal relations of said producers'data relating to the cumulative incidences_n, prevalences_n and durations_n of the same common denominators comprising diagnoses, diseases and disorders and sequelae, relational health-healthcare data, and beneficial effects inherent in the people's, physicians'and scientists'and the animal's in situ and in vivo data; and
the plurality of interactive electronic templates-expressed null relations and causal relations of the producers'data relating to the cumulative incidences, prevalences and durations of the same common denominators comprising diagnoses, diseases and disorders and sequelae, relational health-healthcare data, and beneficial effects inherent in all people's, physicians'and scientists'and the animal's in situ and in vivo data, wherein n =thousands to millions (see below) which are highest confidence levels.

The plurality of interactive comparative graphs for expressing and demonstrating the null relations for enlightening people on the null relations, comprising:
the producers'data and related quantities, qualities and durations, and
the interactive graphs of the random, non prevalent or uncommon diagnoses_m, diseases_m and disorders_m and sequelae_m, relational health-healthcare data_m, and beneficial effects_m, wherein m=or less than hundreds, compiled over minutes, hours, days, decades and centuries, comprising random, non prevalent or uncommon diagnoses, diseases and disorders and sequelae, relational health-healthcare data, and beneficial effects in the plurality of interactive electronic templates-expressed null relations and causal relations in some people's, physicians'and scientists'and animal's in situ and in vivo data, in and shared among the storage memories of the billions of abiotic intelligence-created networked communication devices;
the abiotic intelligence-created networked communication devices have the innovative computer programs including the commercial software including commercial statistical and actuarial software for correlating and statistically accounting the producers'data and the random, non prevalent or uncommon diagnoses_m, diseases_m and disorders_m and sequelae_m, relational health-healthcare data_m, and beneficial effects_m, with confidence levels, in and shared among the storage memories.

Further to the aforementioned interactive comparative graphs for expressing and demonstrating the null relations, the plurality of interactive comparative graphs for expressing and demonstrating the causal relations comprising:
the producers'data including quantities, qualities and durations;
the vertical Y-axis comprising cumulative incidences, prevalences and durations_n of same common denominators, wherein n=thousands to millions, compiled over minutes, hours, days, decades and centuries, comprising same common diagnoses; same common, diseases and disorders and sequelae; same common diagnoses-specific chronologically ordered sequences of relational chief complaints, injuries, symptoms, signs, review of systems and physical examinations; and same common beneficial effects in the plurality of interactive electronic templates-expressed null relations and causal relations in the in billions of people's, physicians'and scientists'and animal's in situ and in vivo data, in and shared among the storage memories of the billions of abiotic intelligence-created networked communication devices;
the horizontal X-axis comprising:
same common diagnoses;
same common, diseases and disorders and sequelae;
same common diagnoses-specific chronologically ordered sequences of relational chief complaints, injuries, symptoms, signs, review of systems and physical examinations; and
same common beneficial effects; and
the interactive comparative polymodal graphs comprising all or any combinations thereof:
multimodal distributions graphs comprising a plurality of peaks of individual cumulative incidences, prevalences and durations of the cumulative incidences, prevalences and durations_n a plurality of common diagnoses;
quad modal distribution graphs comprising four peaks of individual cumulative incidences, prevalences and durations of the cumulative incidences, prevalences and durations_n of four common diagnoses;
trimodal distribution graphs comprising three peaks of individual cumulative incidences, prevalences and durations of the cumulative incidences, prevalences and durations of three common diagnoses;
bimodal distribution graphs comprising two peaks of individual cumulative incidences, prevalences and durations of the cumulative incidences, prevalences and durations_n of two common diagnoses;
unimodal distribution graphs comprising one peak of compilation of the cumulative incidences, prevalences and durations of the cumulative incidences, prevalences and durations_n of one common diagnoses.

Histograms are analogous to said plurality of interactive comparative graphs for expressing and demonstrating the null relations for enlightening people on the null relations.

1-15. (canceled)

16. An abiotic intelligence-rendered networked communication devices for creating, establishing and expressing null relations, causal relations and beneficial effects of producers'same data, comprising producers'data, on cumulative incidences, prevalences and durations of diagnoses-specific same in situ and in vivo relational health-healthcare data (cumulative diagnoses-specific same CORD) relating to diagnoses, diseases and disorders and sequelae of people, physicians, scientists and animals, collectively known herein as the abiotic intelligence-rendered longevity (AIR Longevity), comprising:
the abiotic intelligence comprises networked servers, smartphones, laptops and desktops comprise the touchscreens and viewing screens, cameras, microphones, the processor units attaching to storage memories for storing an innovative computer programs including commercial software for instructing the processor units for creating:
a plurality of interactive electronic templates for creating, establishing and expressing the null relations, causal relations and beneficial effects, in and share among the touchscreens and viewing screens; and
a plurality of interactive comparative graphs for expressing and demonstrating said null relations and causal relations and beneficial effects wherefore enhancing health, longevity and preservations of monies of people and animals, in and share among the touchscreens and viewing screens.

17. The AIR Longevity, according to claim 16, wherein the producers'data comprise:
the producers'quantities, qualities and durations of chemicals, foods including nutrition and chemical contents and facts, chemical additives, pharmaceuticals and fluids, relating to the processor units of the producers'networked communication devices, in and share among the storage memories.

18. The AIR Longevity, according to claim 16, wherein the producers'same data comprise:
individual same quantities, qualities and durations of individual same chemicals, individual same foods including nutrition and chemical contents and facts, individual same chemical additives, individual same pharmaceuticals and fluids, relating to the processor units of the producers'networked communication devices, in and share among the storage memories;

19. The AIR Longevity, according to claim 16, wherein the diagnoses-specific same CORD comprise:
the diagnoses-specific chronologically ordered sequences of relational individual same in situ and in vivo chief complaints, individual same in situ and in vivo injuries, individual same in situ and in vivo symptoms, individual same in situ and in vivo signs, individual same in situ and in vivo review of systems and individual same in situ and in vivo physical examinations, relating to diagnoses, diseases and disorders and sequelae. relating to the processor units of the people's, physicians'and scientists'networked communication devices, in and share among the storage memories.

20. The AIR Longevity, according to claim 16, wherein the plurality of interactive electronic templates for creating, establishing and expressing the null relations, causal relations and beneficial effects, comprising:
a plurality of interactive electronic templates for letting the producers inputting and compiling the producers'data as the producers'same data, relating to the processor units of the producers'networked communication devices, in and share among the storage memories;
a plurality of interactive electronic templates for displaying the interactive list of interactive and selectable producers'same data;
a plurality of interactive electronic templates for letting the people, physicians and scientists inputting, creating thence compiling the people's, physicians'and scientists'and the animal's in situ and in vivo relational health-healthcare data as the cumulative incidences, prevalences and durations of diagnoses-specific same in situ and in vivo relational health-healthcare data relating to the diagnoses, diseases and disorders and sequelae, relating to said people's, physicians'and scientists'networked communication devices, in and share among the storage memories;
a plurality of interactive electronic templates for displaying the interactive list of interactive and selectable cumulative incidences, prevalences and durations of diagnoses-specific same CORD;
said plurality of interactive electronic templates for letting the people, physicians and scientists selecting thence relating said interactive and selectable the producers'same data relating to the processor units of the producers'networked communication devices, and said interactive and selectable cumulative incidences, prevalences and durations of diagnoses-specific same in situ and in vivo relational health-healthcare data relating to said people's, physicians'and scientists'networked communication devices, wherefore
creating thence expressing the null relations, causal relations and beneficial effects of the producers'same data on the cumulative incidences, prevalences and durations of diagnoses-specific same in situ and in vivo relational health-healthcare data.

21. The null relations, causal relations and beneficial effects, according to claim 20, comprise relational data:
the producers'same data, comprising individual quantities, qualities and durations of individual same chemicals, individual same foods including nutrition and chemical contents and facts, individual same chemical additives, individual same pharmaceuticals and fluids, relating to the processor units of the producers'networked communication devices; and
the cumulative incidences, prevalences and durations of diagnoses-specific same CORD, comprising individual same in situ and in vivo chief complaints, individual same in situ and in vivo injuries, individual same in situ and in vivo symptoms, individual same in situ and in vivo signs, individual same in situ and in vivo review of systems and individual same in situ and in vivo physical examinations, relating to diagnoses, diseases and disorders and sequelae, relating to the processor units of the people's, physicians'and scientists'networked communication devices in the cumulative incidences, prevalences and durations of diagnoses-specific same CORD, in and share among the storage memories.

22. The null relations, according to claim 20, comprising:
1. the producers'same data unrelating to individual same chief complaints, individual same injuries, individual same symptoms, individual same signs, individual same review of systems and individual same physical examinations, in the cumulative incidences, prevalences and durations of diagnoses-specific same CORD; and
2. the producers'same data relating to a plurality albeit not most of the individual same chief complaints, individual same injuries, individual same symptoms, individual same signs, individual same review of systems and individual same physical examinations, in the cumulative incidences, prevalences and durations of diagnoses-specific same CORD.

23. The causal relations and beneficial effects, according to claim 20, comprising:
1. the producers'same data relating to individual same chief complaints, individual same injuries, individual same symptoms, individual same signs, individual same review of systems and individual same physical examinations, in the cumulative incidences, prevalences and durations of diagnoses-specific same CORD;
2. the producers'same data relating to the attenuations of individual same chief complaints, individual same injuries, individual same symptoms, individual same signs, individual same review of systems and individual same physical examinations, in the cumulative incidences, prevalences and durations of diagnoses-specific same CORD;
3. the producers'same data relating to the attenuations of the plurality albeit not all of the individual same chief complaints, individual same injuries, individual same symptoms, individual same signs, individual same review of systems and individual same physical examinations, in the cumulative incidences, prevalences and durations of diagnoses-specific same CORD;
4. the producers'same data relating to the resolutions of the plurality of individual same chief complaints, individual same injuries, individual same symptoms, individual same signs, individual same review of systems and individual same physical examinations, in the cumulative incidences, prevalences and durations of diagnoses-specific same CORD; and
5. the producers'same data relating to the resolutions of the plurality albeit not all of the individual same chief complaints, individual same injuries, individual same symptoms, individual same signs, individual same review of systems and individual same physical examinations, in the cumulative incidences, prevalences and durations of diagnoses-specific same CORD.

24. The AIR Longevity, according to claim 16, wherein the plurality of interactive comparative graphs comprise interactive comparative polymodal graphs, which are the multimodal distributions graphs comprise:
1. the null relations comprise:
horizontal X-axes comprise the producers'same data;
vertical Y-axes comprise the incidences and prevalences, horizontal z-axes comprise the durations, and the multimodal distributions curves comprise cumulative incidences, prevalences and durations of diagnoses-specific as follow:
individual same chief complaints, individual same injuries, individual same symptoms, individual same signs, individual same review of systems and individual same physical examinations, in the cumulative incidences, prevalences and durations of diagnoses-specific same CORD and
2. the causal relations and beneficial effects comprise:
horizontal X-axes comprise the producers'same data; and
vertical Y-axes comprise the incidences and prevalences, horizontal z-axes comprise the durations, and the multimodal distributions curves
comprise the cumulative incidences, prevalences and durations of diagnoses-specific same CORD as follow:
individual same chief complaints, individual same injuries, individual same symptoms, individual same signs, individual same review of systems and individual same physical examinations, in the cumulative incidences, prevalences and durations of diagnoses-specific same CORD.

25. The AIR Longevity, according to claim 16, wherein the plurality of interactive comparative graphs comprise interactive comparative polymodal graphs, which are the multiple comparative vertical bars comprise:
1. the null relations comprise:
horizontal X-axes comprise the producers'same data;
vertical Y-axes comprise the incidences and prevalences, horizontal z-axes comprise the durations, and the multiple comparative vertical bars comprise the cumulative incidences, prevalences and durations of diagnoses-specific same CORD as follow:
individual same chief complaints, individual same injuries, individual same symptoms, individual same signs, individual same review of systems and individual same physical examinations, in the cumulative incidences, prevalences and durations of diagnoses-specific same CORD; and
2. the causal relations and beneficial effects comprise:
horizontal X-axes comprise the producers'same data;
vertical Y-axes comprise the incidences and prevalences, horizontal z-axes comprise the durations, and the multiple comparative vertical bars comprise the cumulative incidences, prevalences and durations of diagnoses-specific same CORD as follow:
individual same chief complaints, individual same injuries, individual same symptoms, individual same signs, individual same review of systems and individual same physical examinations, in the cumulative incidences, prevalences and durations of diagnoses-specific same CORD.

26. In the AIR Longevity, a method of the abiotic intelligence networking networked communication devices creating, establishing and expressing null relations, causal relations and beneficial effects of producers'same data on the cumulative incidences, prevalences and durations of diagnoses-specific same in situ and in vivo relational health-healthcare data (cumulative incidences, prevalences and durations of diagnoses-specific same CORD) relating to diagnoses, diseases and disorders and sequelae of people, physicians, scientists and animals, whereby
the abiotic intelligence comprises networked servers, smartphones, laptops and desktops comprise the touchscreens and viewing screens, cameras, microphones, the processor units attaching to storage memories storing an innovative computer programs including commercial software instructing the processor units creating:
the plurality of interactive electronic templates creating, establishing and expressing the null relations, causal relations and beneficial effects, in and share among the touchscreens and viewing screens; and
the plurality of interactive comparative graphs expressing and demonstrating said null relations and causal relations and beneficial effects which enhancing health, longevity and preservations of monies of people and animals, in and share among the touchscreens and viewing screens.

27. The method, according to claim 26, whereby the plurality of interactive electronic templates creating, establishing and expressing the null relations, causal relations and beneficial effects, comprising the process of the innovative computer programs including commercial software instructing the processor units creating
the plurality of interactive electronic templates letting the producers inputting and compiling the producers'data as the producers'same data relating to the processor units of the producers'networked communication devices, in and share among the storage memories;
the plurality of interactive electronic templates displaying the interactive list of interactive and selectable producers'same data;
the plurality of interactive electronic templates letting the people, physicians and scientists inputting, creating thence compiling the people's, physicians'and scientists'and the animal's in situ and in vivo relational health-healthcare data, in and share among the storage memories;
the plurality of interactive electronic templates letting the people, physicians and scientists inputting, selecting thence compiling the people's, physicians'and scientists'and the animal's in situ and in vivo relational health-healthcare data as the cumulative incidences, prevalences and durations of diagnoses-specific same in situ and in vivo relational health-healthcare data relating to the diagnoses, diseases and disorders and sequelae relating to said people's, physicians'and scientists'networked communication devices, in and share among the storage memories;
the plurality of interactive electronic templates displaying the interactive list of interactive and selectable cumulative incidences, prevalences and durations of diagnoses-specific same CORD;
said plurality of interactive electronic templates letting the people, physicians and scientists selecting thence relating said interactive and selectable producers'same data relating to the processor units of the producers'networked communication devices, and said interactive and selectable cumulative incidences, prevalences and durations of diagnoses-specific same in situ and in vivo relational health-healthcare data relating to said people's, physicians'and scientists'networked communication devices, thereby
creating thence expressing the null relations, causal relations and beneficial effects of the producers'same data on the cumulative incidences, prevalences and durations of diagnoses-specific same in situ and in vivo relational health-healthcare data.

28. The plurality of interactive electronic templates letting the people, physicians and scientists selecting thence relating the interactive and selectable producers'same data and the interactive and selectable diagnoses-specific CORD, according to claim 27, comprising the process of the innovative computer programs including commercial software instructing the processor units to letting the people, physicians and scientists to selecting, relating thence compiling relational data comprise:
interactive and selectable individual same quantities, qualities and durations of individual same chemicals, same individual foods including nutrition and chemical contents and facts, individual same chemical additives, individual same pharmaceuticals and fluids, relating to the processor units of the producers'networked communication devices; and
interactive and selectable individual same in situ and in vivo chief complaints, individual same in situ and in vivo injuries, individual same in situ and in vivo symptoms, individual same in situ and in vivo signs, individual same in situ and in vivo review of systems and individual same in situ and in vivo physical examinations relating to diagnoses, diseases and disorders and sequelae, relating to the processor units of said people's, physicians'and scientists'networked communication devices, in the cumulative incidences, prevalences and durations of diagnoses-specific same CORD, thereby
creating thence expressing the null relations, causal relations and beneficial effects of the producers'same data on the cumulative incidences, prevalences and durations of the diagnoses-specific same in situ and in vivo relational health-healthcare data, relating to said processor units of said people's, physicians'and scientists'networked communication devices, in and share among the storage memories.

29. The method, according to claim 26, whereby the process of the plurality of interactive electronic templates creating, establishing and expressing the null relations, according to claim 26, comprising the process of the innovative computer programs including commercial software instructing the processor units effecting,
according to said plurality of interactive electronic templates letting the people, physicians and scientists selecting thence relating the interactive and selectable producers'same data and the interactive and selectable cumulative incidences, prevalences and durations of diagnoses-specific same in situ and in vivo relational health-healthcare data,
1. the producers'same data unrelating to individual same chief complaints, individual same injuries, individual same symptoms, individual same signs, individual same review of systems and individual same physical examinations, in the cumulative incidences, prevalences and durations of diagnoses-specific same CORD; and
2. the producers'same data relating to a plurality albeit not most of the individual same chief complaints, individual same injuries, individual same symptoms, individual same signs, individual same review of systems and individual same physical examinations, in the cumulative incidences, prevalences and durations of diagnoses-specific same CORD.

30. The method, according to claim 26, whereby the process of the plurality of interactive electronic templates creating, establishing and expressing the causal relations and beneficial effects, comprising the process of the innovative computer programs including commercial software instructing the processor units effecting,
according to said plurality of interactive electronic templates letting the people, physicians and scientists selecting thence relating the interactive and selectable producers'same data and the interactive and selectable cumulative incidences, prevalences and durations of diagnoses-specific same in situ and in vivo relational health-healthcare data,
1. the producers'same data relating to individual same chief complaints, individual same injuries, individual same symptoms, individual same signs, individual same review of systems and individual same physical examinations, in the cumulative incidences, prevalences and durations of diagnoses-specific same CORD;
2. the producers'same data relating to the attenuations of individual same chief complaints, individual same injuries, individual same symptoms, individual same signs, individual same review of systems and individual same physical examinations, in the cumulative incidences, prevalences and durations of diagnoses-specific same CORD;
3. the producers'same data relating to the attenuations of the plurality albeit not all of the individual same chief complaints, individual same injuries, individual same symptoms, individual same signs, individual same review of systems and individual same physical examinations, in the cumulative incidences, prevalences and durations of diagnoses-specific same CORD;
4. the producers'same data relating to the resolutions of the plurality of individual same chief complaints, individual same injuries, individual same symptoms, individual same signs, individual same review of systems and individual same physical examinations, in the cumulative incidences, prevalences and durations of diagnoses-specific same CORD; and
5. the producers'same data relating to the resolutions of the plurality albeit not all of the individual same chief complaints, individual same injuries, individual same symptoms, individual same signs, individual same review of systems and individual same physical examinations, in the cumulative incidences, prevalences and durations of diagnoses-specific same CORD.

31. The method, according to claim 26, whereby the process of the plurality of interactive comparative graphs expressing and demonstrating the plurality of interactive electronic templates-expressed null relations and causal relations and beneficial effects comprises the process of the innovative computer programs including commercial software instructing the processor units creating the multimodal distributions graphs comprise:
1. the null relations comprise:
horizontal X-axes comprise the producers'same data;
vertical Y-axes comprise the incidences and prevalences, horizontal z-axes comprise the durations, and the multimodal distributions curves comprise the cumulative incidences, prevalences and durations of diagnoses-specific same CORD as follow:
individual same chief complaints, individual same injuries, individual same symptoms, individual same signs, individual same review of systems and individual same physical examinations, in the cumulative incidences, prevalences and durations of diagnoses-specific same CORD and
2. the causal relations and beneficial effects comprise:
horizontal X-axes comprise the producers'same data; and
vertical Y-axes comprise the incidences and prevalences, horizontal z-axes comprise the durations, and the multimodal distributions curves
comprise the cumulative incidences, prevalences and durations of diagnoses-specific same CORD as follow:
individual same chief complaints, individual same injuries, individual same symptoms, individual same signs, individual same review of systems and individual same physical examinations, in the cumulative incidences, prevalences and durations of diagnoses-specific same CORD.

32. The method, according to claim 26, whereby the process of the plurality of interactive comparative graphs expressing and demonstrating the plurality of interactive electronic templates-expressed null relations and causal relations and beneficial effects comprises the process of the innovative computer programs including commercial software instructing the processor units creating the multiple comparative vertical bars comprise:
1. the null relations comprise:
horizontal X-axes comprise the producers'same data;
vertical Y-axes comprise the incidences and prevalences, horizontal z-axes comprise the durations, and the multiple comparative vertical bars comprise the cumulative incidences, prevalences and durations of diagnoses-specific same CORD as follow:
individual same chief complaints, individual same injuries, individual same symptoms, individual same signs, individual same review of systems and individual same physical examinations, in the cumulative incidences, prevalences and durations of diagnoses-specific same CORD; and
2. the causal relations and beneficial effects comprise:
horizontal X-axes comprise the producers'same data;
vertical Y-axes comprise the incidences and prevalences, horizontal z-axes comprise the durations, and the multiple comparative vertical bars comprise the cumulative incidences,
prevalences and durations of diagnoses-specific same CORD as follow:
individual same chief complaints, individual same injuries, individual same symptoms, individual same signs, individual same review of systems and individual same physical examinations, in the cumulative incidences, prevalences and durations of diagnoses-specific same CORD.
